(19) United States
(12) Patent Application Publication
(10) Pub. No.: US 2025/0339062 A1
(43) Pub. Date: Nov. 6, 2025

(54) SUBCUTANEOUS MEDICAL DEVICE SYSTEM

(71) Applicant: Unomedical A/S, Lejre (DK)

(72) Inventors: Chih-Ho Chen, Taipei City (TW); Hung-Hsiang Chen, Boston, MA (US)

(21) Appl. No.: 19/266,250

(22) Filed: Jul. 11, 2025

Related U.S. Application Data
(63) Continuation of application No. PCT/EP2024/050554, filed on Jan. 11, 2024.
(60) Provisional application No. 63/438,826, filed on Jan. 13, 2023.
(60) Provisional application No. 63/438,838, filed on Jan. 13, 2023.

(30) Foreign Application Priority Data
Mar. 1, 2023 (GB) 2302987.9
Mar. 1, 2023 (GB) 2302988.7

(51) Int. Cl. A61B 5/145; A61B 5/00; A61B 90/00

(52) U.S. Cl. CPC A61B 5/14532; A61B 5/14503; A61B 5/6833; A61B 90/08; A61B 2090/0808; A61B 2560/06

(57) ABSTRACT

The present invention provides a subcutaneous medical device system, comprising an interface patch and a subcutaneous medical device having an adhesive surface and a needle for piercing a patient's skin. The interface patch comprises: a proximal side having an adhesive region for attaching the interface patch to the patient's skin; and a distal side having a locating edge for aligning an applicator operable to apply the subcutaneous medical device to the distal side of the interface patch.

The invention relates generally to a subcutaneous medical device system.

### BACKGROUND

Subcutaneous medical devices, such as glucose sensors and infusion sets, are worn by patients for prolonged periods of time (e.g., days or weeks) and include a needle that is subcutaneously positioned, e.g., to detect blood glucose or deliver insulin. These devices typically have a small and discreet housing that is attached to a patient by an adhesive patch. The adhesive patch is attached to the housing and is located between the housing and the skin when applied, and acts to seal the puncture site of the needle.

As these devices are worn for prolonged periods of time, the adhesive patch typically has a strong, water-resistant adhesive to prevent the device from moving or coming detached and to maintain protection of the skin puncture site.

However, removal of these devices, particularly the adhesive patch, can cause skin irritation and damage, such as medical adhesive related skin injuries. This can include tension injuries caused by pulling the skin during removal, blisters, skin tears, skin stripping, irritant contact dermatitis, maceration, and folliculitis.

Additionally, some patients wear an additional covering dressing that covers the device (housing and adhesive patch) to protect it from knocks, water and dirt.

These additional dressings are applied separately to the device and are also adhered to the skin and also cause skin irritation and damage.

It would be desirable to provide an apparatus that addresses at least some of the above problems.

### SUMMARY OF THE INVENTION

In accordance with the present invention there is provided a subcutaneous medical device system according to the appended claims.

According to the invention, there is provided a subcutaneous medical device system, comprising an interface patch and a subcutaneous medical device having an adhesive surface and a needle for piercing a patient's skin, wherein the interface patch comprises: a proximal side having an adhesive region for attaching the interface patch to the patient's skin; and a distal side having a locating edge for aligning an applicator operable to apply the subcutaneous medical device to the distal side of the interface patch.

Thus, the locating edge on the distal side of the interface patch ensures alignment between the interface patch and the applicator, and thus alignment between the interface patch and the subcutaneous medical device applied by the applicator. This is advantageous because even when the interface patch is applied to the patient's skin in a region of the body that is not visible to the patient, the applicator can be more easily aligned with the interface patch to precisely position the subcutaneous medical device when it is applied.

In examples, the interface patch further comprises an aperture positioned to receive the needle of the subcutaneous medical device. In examples, the locating edge aligns the needle of the subcutaneous medical device with the aperture of the interface patch.

In other examples, the needle of the subcutaneous medical device pierces the interface patch as it is applied by the applicator. In examples, the interface patch comprises a pierceable region, for example a thinned region or a region having a different material. In examples, the locating edge aligns the needle of the subcutaneous medical device with the pierceable region of the interface patch.

In examples, the locating edge comprises a step. The steps provides a surface for engagement with the applicator, to better align the applicator for application of the subcutaneous medical device. In examples, the applicator comprises an end face positionable on the interface patch, and the locating edge may be shaped to match the end face.

In examples, the distal side comprises a first portion and a second portion. In examples the locating edge is provided between the first portion and the second portion.

In examples, the second portion is provided outward of the first portion.

In examples, the first portion is raised relative to the second portion.

In examples, the second portion is raised relative to the first portion. In examples, the first portion is central on the interface patch. The difference in heights between the first portion and the second portion are configured such that the locating edge can engage with a different part of the applicator.

In some examples, the first portion is central about an aperture on the interface patch if provided. In examples where the second portion is provided outward of the first portion and is raised relative to the first portion, the first portion is recessed such that the recessed first portion holds the applicator in alignment with the interface patch.

In other examples, the first portion is central about a pierceable region on the interface patch if provided.

In examples, the first portion is concentric with the second portion.

In examples, the interface patch further comprises a third portion outward of the second portion. In examples, an interface between the second portion and the third portion comprises an outer locating edge. This allows a locating edge to be provided for aligning a first applicator, and an outer locating edge to be provided for aligning a second applicator having a larger size than the first applicator. Accordingly, the interface patch may be useable with more than one type of applicator.

In examples, the height of the third portion is substantially the same as the height of the first portion.

In examples, the third portion and the first portion are concentric.

In examples, the third portion and the second portion are concentric.

In examples, the second portion is raised relative to the third portion.

In examples, the locating edge is circular. Other shapes are also envisaged that are selected to correspond with the shape of the applicator, for alignment of the applicator with the interface patch.

In examples, the interface patch comprises a support attached to the distal side of the interface patch, the support comprising the locating edge. In examples, the support is removably attached to the interface patch.

In examples, the support is rigid. In examples, the support is more rigid than the interface patch. This is advantageous because the interface patch is held in a flat position during the application of the interface patch to the patient's skin. In examples, the support has an adhesive which remains on the support after the support is removed from the skin. In examples, a single use adhesive is used to attach the support to the interface patch. In examples, the adhesive is a single use, non-tacky adhesive. In examples, the support is attached to a pull tab for separating the support and the interface patch.

In examples, the subcutaneous medical device further comprises an applicator operable to apply the subcutaneous medical device to the interface patch. In examples, the applicator comprises an end face positionable on the interface patch, and wherein the locating edge is shaped to match the end face. In examples, the end face of the applicator is circular and the locating edge is circular.

In examples, the subcutaneous medical device further comprises a cover positionable over the subcutaneous medical device and comprising a concave portion for accommodating the subcutaneous medical device. In examples, the cover comprises a flat portion outward of the concave portion in engagement with the second portion of the interface patch.

According to the invention, there is provided a method of using the subcutaneous medical device system. The method comprises: applying the interface patch to the patient's skin, aligning the applicator with the locating edge on the distal side of the interface patch, and operating the applicator to apply the subcutaneous medical device to the distal side of the interface patch.

According to the invention, there is provided an interface patch for a subcutaneous medical device having an adhesive surface and a needle for piercing a patient's skin, wherein the interface patch comprises: a proximal side having an adhesive region for attaching the interface patch to the patient's skin; and a distal side having a locating edge for aligning an applicator operable to apply the subcutaneous medical device to the distal side of the interface patch.

Thus, the locating edge on the distal side of the interface patch ensures alignment between the interface patch and the applicator, and thus alignment between the interface patch and the subcutaneous medical device applied by the applicator. This is advantageous because even when the interface patch is applied to the patient's skin in a region of the body that is not visible to the patient, the applicator can be more easily aligned with the interface patch to precisely position the subcutaneous medical device when it is applied.

In examples, the interface patch further comprises an aperture positioned to receive the needle of the subcutaneous medical device. In examples, the locating edge aligns the needle of the subcutaneous medical device with the aperture of the interface patch.

In other examples, the needle of the subcutaneous medical device pierces the interface patch as it is applied by the applicator. In examples, the interface patch comprises a pierceable region, for example a thinned region or a region having a different material. In examples, the locating edge aligns the needle of the subcutaneous medical device with the pierceable region of the interface patch.

In examples, the locating edge comprises a step. The steps provides a surface for engagement with the applicator, to better align the applicator for application of the subcutaneous medical device. In examples, the applicator comprises an end face positionable on the interface patch, and the locating edge may be shaped to match the end face.

In examples, the distal side comprises a first portion and a second portion. In examples the locating edge is provided between the first portion and the second portion.

In examples, the second portion is provided outward of the first portion.

In examples, the first portion is raised relative to the second portion.

In examples, the second portion is raised relative to the first portion. In examples, the first portion is central on the interface patch. The difference in heights between the first portion and the second portion are configured such that the locating edge can engage with a different part of the applicator.

In some examples, the first portion is central about an aperture on the interface patch if provided. In examples where the second portion is provided outward of the first portion and is raised relative to the first portion, the first portion is recessed such that the recessed first portion holds the applicator in alignment with the interface patch.

In other examples, the first portion is central about a pierceable region on the interface patch if provided.

In examples, the first portion is concentric with the second portion.

In examples, the interface patch further comprises a third portion outward of the second portion. In examples, an interface between the second portion and the third portion comprises an outer locating edge. This allows a locating edge to be provided for aligning a first applicator, and an outer locating edge to be provided for aligning a second applicator having a larger size than the first applicator. Accordingly, the interface patch may be useable with more than one type of applicator.

In examples, the height of the third portion is substantially the same as the height of the first portion.

In examples, the third portion and the first portion are concentric.

In examples, the third portion and the second portion are concentric.

In examples, the second portion is raised relative to the third portion.

In examples, the locating edge is circular. Other shapes are also envisaged that are selected to correspond with the shape of the applicator, for alignment of the applicator with the interface patch.

In examples, the interface patch comprises a support attached to the distal side of the interface patch, the support comprising the locating edge. In examples, the support is removably attached to the interface patch.

In examples, the support is rigid. In examples, the support is more rigid than the interface patch. This is advantageous because the interface patch is held in a flat position during the application of the interface patch to the patient's skin. In examples, the support has an adhesive which remains on the support after the support is removed from the skin. In examples, a single use adhesive is used to attach the support to the interface patch. In examples, the adhesive is a single use, non-tacky adhesive. In examples, the support is attached to a pull tab for separating the support and the interface patch.

According to the invention, there is provided a subcutaneous medical device system for attaching to a patient's skin, comprising: a subcutaneous medical device comprising an adhesive surface and a needle for piercing a patient's skin, and an interface patch as described above.

In examples, the subcutaneous medical device further comprises an applicator operable to apply the subcutaneous medical device to the interface patch. In examples, the applicator comprises an end face positionable on the interface patch, and wherein the locating edge is shaped to match the end face. In examples, the end face of the applicator is circular and the locating edge is circular.

In examples, the subcutaneous medical device further comprises a cover positionable over the subcutaneous medical device and comprising a concave portion for accommodating the subcutaneous medical device. In examples, the cover comprises a flat portion outward of the concave portion in engagement with the second portion of the interface patch.

According to the invention, there is provided a method of using the interface patch to attach the subcutaneous medical device to a patient's skin. The method comprises: applying the interface patch to the patient's skin, aligning the applicator with the locating edge on the distal side of the interface patch, and operating the applicator to apply the subcutaneous medical device to the distal side of the interface patch.

According to the invention, there is provided a subcutaneous medical device system, comprising: a subcutaneous medical device comprising an adhesive surface and a needle for piercing a patient's skin; an applicator operable to apply the subcutaneous medical device; and an interface patch comprising a proximal side having an adhesive for attaching the interface patch to the patient's skin, and a distal side to which the adhesive surface of the subcutaneous medical device is attachable, wherein the interface patch is attachable to the applicator before operation of the applicator.

Thus, the applicator can be attached to the interface patch before the interface patch is attached to the patient's skin. This removes the need to align the applicator to the interface patch after the interface patch is attached to the patient's skin, and simplifies application of the interface patch and subcutaneous medical device because they are applied at the same time. This is particularly advantageous when the subcutaneous medical device is to be applied to a region of the skin that is not visible to the patient or is hard to reach. In some examples, the interface patch is pre-assembled and attached to the applicator and provided to the patient as a single assembly. In other examples, the interface patch and the applicator are provided to the patient separately and assembled by the patient before application of the interface patch to the patient's skin.

In examples, the interface patch is adherable, or adhered, to the applicator. In examples, the interface patch is adherable, or adhered, to an end face of the applicator. In examples, the distal side of the interface patch comprises an adhesive portion for adhering the interface patch to the applicator. In some examples, the adhesive portion covers substantially the entire distal side of the interface patch. In other examples, the adhesive portion covers an area corresponding to the end face of the applicator. In other examples, the adhesive portion comprises a pattern-applied adhesive. In examples, the pattern-applied adhesive has a line pattern. In examples, the pattern-applied adhesive has a grid pattern.

In examples, the applicator comprises an end face arranged to contact the distal side of the interface patch. In examples, the end face of the applicator is adherable, or adhered, onto the distal side of the interface patch.

In examples, the interface patch comprises a support structure for attaching the interface patch to the applicator. In examples, the support structure comprises an outer portion. In examples, the outer portion of the support structure is coincident with the edge of the interface patch, particularly the peripheral edge of the interface patch.

In examples, the support structure comprises an inner portion. In examples, the inner portion is attachable to the applicator.

In examples, the inner portion of the support structure is shaped to match the applicator. In examples, the inner portion of the support structure has a circular shape.

In examples, the inner portion of the support structure comprises at least one support member configured for engaging the applicator. In examples, the support member resiliently biases against the applicator during use. By providing one or more support members that resiliently bias against the applicator during use, the support members urge against and hold the applicator in place, and the applicator can be removed after use.

In examples, the interface patch comprises an aperture aligned with the needle of the subcutaneous medical device when the interface patch is attached to the applicator. In examples, the aperture is arranged substantially in the center of the interface patch.

In other examples, the interface patch comprises a pierceable region that is pierceable by the needle of the subcutaneous medical device as it is applied to the interface patch. In examples, the pierceable region comprises thinned section of the interface patch, or a different material to the rest of the interface patch. In examples, the pierceable region is arranged substantially in the center of the interface patch.

In examples, the interface patch has a generally circular shape. In other examples, the interface patch has an oval shape.

In examples, the subcutaneous medical device is a glucose sensor. In some examples, the glucose sensor is an intermittent glucose sensor. In other examples, the glucose sensor is a continuous glucose sensor.

In some examples, the subcutaneous medical device is an infusion set.

According to the invention, there is also provided a method of using the subcutaneous medical device system. The method comprises: providing an applicator with a subcutaneous medical device and an interface patch attached to the applicator, positioning the interface patch on the patient's skin, and operating the applicator to apply the subcutaneous medical device to the interface patch.

According to the invention, there is also provided a method of using the subcutaneous medical device system. The method comprises: providing an applicator with a subcutaneous medical device, attaching an interface patch to the applicator, positioning the interface patch on the patient's skin, and operating the applicator to apply the subcutaneous medical device to the interface patch.

According to the invention, there is also provided a support structure for attaching an interface patch to an applicator operable to apply a subcutaneous medical device to a patient. The support structure may be as described above. In particular, the support structure may have a first surface for attaching to a distal side of the interface patch (e.g., with an adhesive), and a mount for attachment to the applicator. The support structure may be reusable.

According to the invention, there is also provided an interface patch for a subcutaneous medical device having an adhesive surface and a needle for piercing a patient's skin, wherein the interface patch comprises: a proximal side having an adhesive for attaching the interface patch to the patient's skin, and a distal side to which the adhesive surface of the subcutaneous device is attachable; wherein the interface patch is attachable to an applicator operable to apply the subcutaneous medical device to the patient.

In examples, the interface patch, applicator and subcutaneous medical device may be as described above with respect to the subcutaneous medical device system.

Specifically, in examples, the interface patch is adherable to the applicator as described above. In other examples, the interface patch comprises a support structure for attaching the interface patch to the applicator as described above.

### BRIEF DESCRIPTION OF THE DRAWINGS

Embodiments of the invention are now described, by way of example only, hereinafter with reference to the accompanying drawings, in which:

FIG. 1 illustrates: (a) a subcutaneous medical device system attachable to a patient and an interface patch, (b) a subcutaneous glucose sensor device and an interface patch, (c) a subcutaneous medical device system including a subcutaneous infusion set device and an interface patch, and (d) an applicator for applying a subcutaneous medical device to a patient;

FIG. 2 illustrates an example interface patch (a) in a schematic view, (b) from the distal side, showing a removable release layer on the proximal side, (c) attached to the skin with the proximal side facing the skin, (d) attached to an example subcutaneous medical device to form a subcutaneous medical device system with a cover to be attached to the system and (e) attached to an example subcutaneous medical device to form a subcutaneous medical device system with the cover attached;

FIG. 3 illustrates an example interface patch (a) from the distal side, with a support for aligning an applicator, (b) from the proximal side, (c) from the distal side, attached to a subcutaneous medical device to form a subcutaneous medical device system, with the support attached and (d) attached to a subcutaneous medical device to form a subcutaneous medical device system, with the support removed;

FIG. 4 illustrates an example interface patch (a) with a support structure attached to the interface patch, (b) from the distal side with a support structure attached to the interface patch and including an applicator attached to the support structure, (c) from the proximal side with a support structure attached to the interface patch and including an applicator attached to the support structure and (d) attached to a subcutaneous medical device to form a subcutaneous medical device system;

FIG. 5 illustrates an example interface patch (a) in a schematic view, (b) from the distal side, showing a removable release layer, (c) with the removable release layer removed, (d) attached to an example applicator, (e) with a backing layer removed to attach the interface patch to the skin, attached to a subcutaneous medical device to form a subcutaneous medical device system, (f) attached to the skin with a subcutaneous medical device attached, further with the release layers on the proximal side removed and (g) with a cover attached on the distal side to cover the subcutaneous medical device;

FIG. 6 illustrates (a) an example interface patch with a support structure attached, (b) the example interface patch and support structure, with an applicator attached to the support structure, (c) an assembly including the support structure, applicator and interface patch, viewed from the proximal side, (d) the applicator, support structure and interface patch assembly attached to the skin, with the applicator in a first rest position, (e) the applicator, support structure and interface patch assembly attached to the skin, with the applicator in a second actuated position to apply the subcutaneous medical device, (f) a subcutaneous medical device system with the applicator and support structure removed from the assembly, (g) a subcutaneous medical device system including the interface patch and the subcutaneous medical device, with the support structure attached, and (h) the subcutaneous medical device system with the support structure partially removed.

### DETAILED DESCRIPTION

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first”, “second”, “third” etc. merely indicate that different instances of like objects are being referred to and are not intended to imply that the objects so described must be in a given sequence, either temporally, spatially, in ranking or in any other manner.

Like reference numerals are used to depict like features throughout.

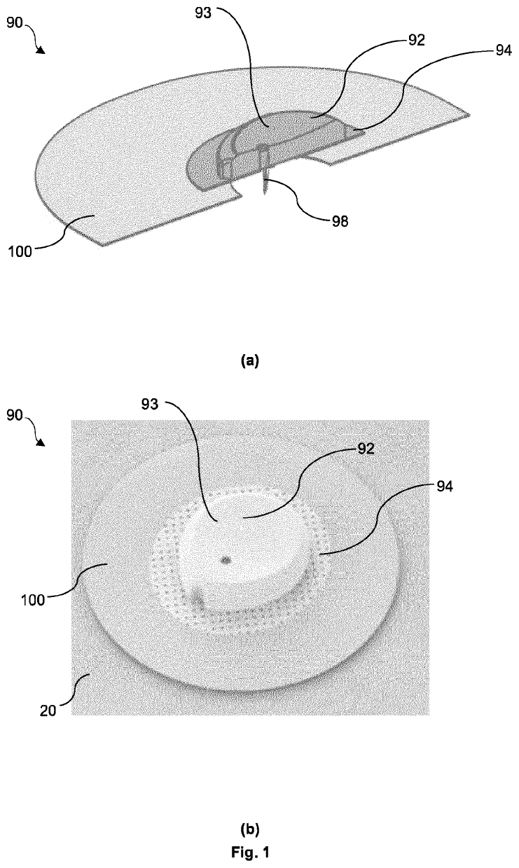

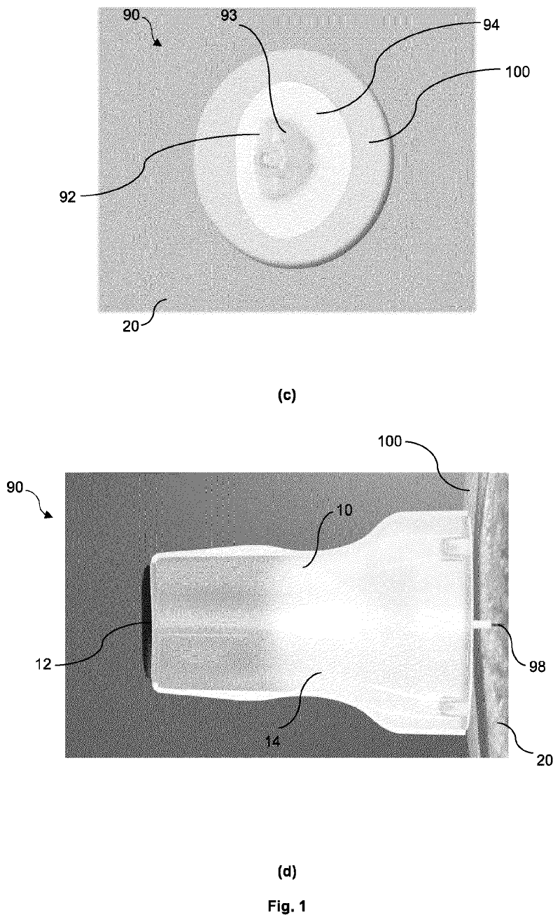

FIGS. 1(a) to 1(c) illustrate example subcutaneous medical device systems 90 that each include a subcutaneous medical device 92. In FIG. 1(b) the subcutaneous medical device 92 is a subcutaneous glucose sensor, and in FIG. 1(c) the subcutaneous medical device 92 is a subcutaneous infusion set of an infusion system. As shown in FIGS. 1(a) to 1(c), in each example the subcutaneous medical device 92 includes a device body 93, an adhesive patch 94 for attaching the device body 93 to a patient's skin 20, and a needle 98 extending from the device body 93 to pierce the patient's skin 20 so that the needle 98 is subcutaneously positioned. The device body 93 is typically rigid and houses other components. The adhesive patch 94 is typically flexible to conform to the skin shape at the attachment site. The adhesive patch 94 has an adhesive surface that faces the skin for attaching the device body 93 to a patient's skin 20, but in other examples the device body 93 itself may have an adhesive surface.

In the example of FIG. 1(b), the subcutaneous glucose sensor 92 includes a sensor adapted to sense a glucose content of blood accessed through the needle 98. The subcutaneous glucose sensor 92 also includes a communications device, such as a communications port or transmitter, for communicating the detected glucose content to an external device. The subcutaneous glucose sensor 92 may be a flash glucose sensor or a continuous glucose monitor.

In the example of FIG. 1(c) the subcutaneous infusion set 92 includes a connector for connecting the needle 98 with an external source of medicament, for example an insulin pump. The subcutaneous infusion set 92 can be used to provide insulin to the patient, through the needle 98, in an intermittent or continuous manner.

FIG. 1(d) illustrates an applicator 10 of the subcutaneous medical device system 90. The applicator 10 is used to apply the subcutaneous medical device 92 to the patient's skin 20. The applicator 10 includes a device body 14 and an actuator 12, in this example a button-operated actuator. In use, the actuator 12 presses the subcutaneous medical device 92 against the patient so that the needle 98 is subcutaneously positioned and the adhesive patch 94 attaches the subcutaneous medical device 92 to the patient. After the subcutaneous medical device 92 has been attached to the patient the applicator 10 is removed.

In other examples, the applicator 10 may have two parts that are collapsed relative to each other to actuate the applicator 10 and press the subcutaneous medical device 10 against the patient.

In various examples, the applicator 10 may be an applicator of the Neria Guard™ system marketed by Convatec™, or the Mio or Mio Advance™ marketed by Medtronic™, or the Autosoft system marketed by Tandem™.

As shown in FIGS. 1(a) to 1(d), the subcutaneous medical device systems 90 include an interface patch 100 disposed between the subcutaneous medical device 92 and the patient's skin 20. In particular, the interface patch 100 is adhered to the patient's skin 20, and the subcutaneous medical device 92 is adhered to the interface patch 100. The interface patch 100 can advantageously improve attachment of the subcutaneous medical device 92 to the patient's skin 20 by providing a better adhesion surface for the adhesive patch 94 than the skin 20. In particular, the interface patch 100 provides a larger surface area in contact with the skin 20 for adhesion. Moreover, the interface patch 100 can reduce skin irritation and damage on removal of the subcutaneous medical device system 90 from the patient. In particular, the larger adhesive surface area provided by the interface patch 100 can use a less strong adhesive, or a pattern-applied adhesive, to make removal of the interface patch 100 from the skin 20 easier without reducing the overall attachment force for the subcutaneous medical device 92.

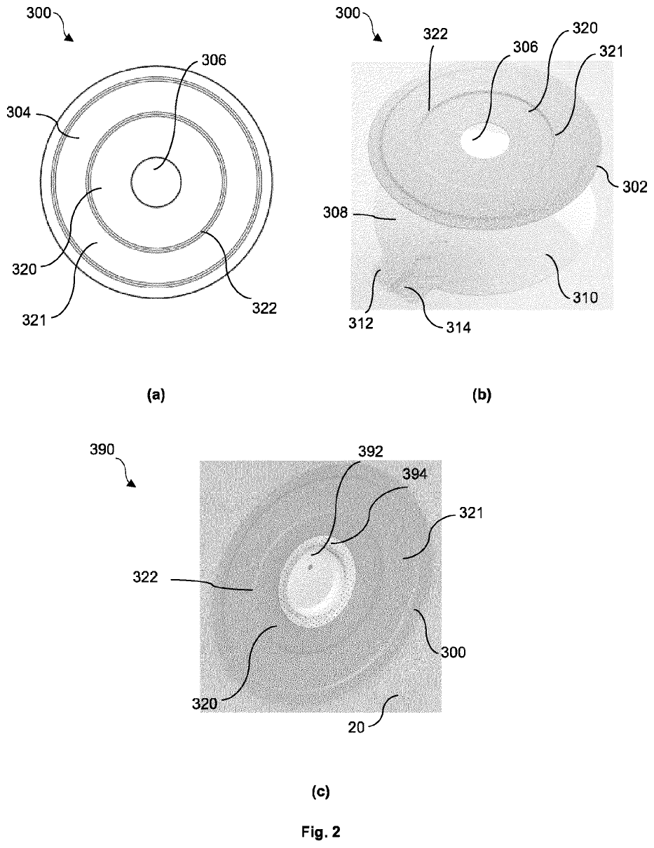

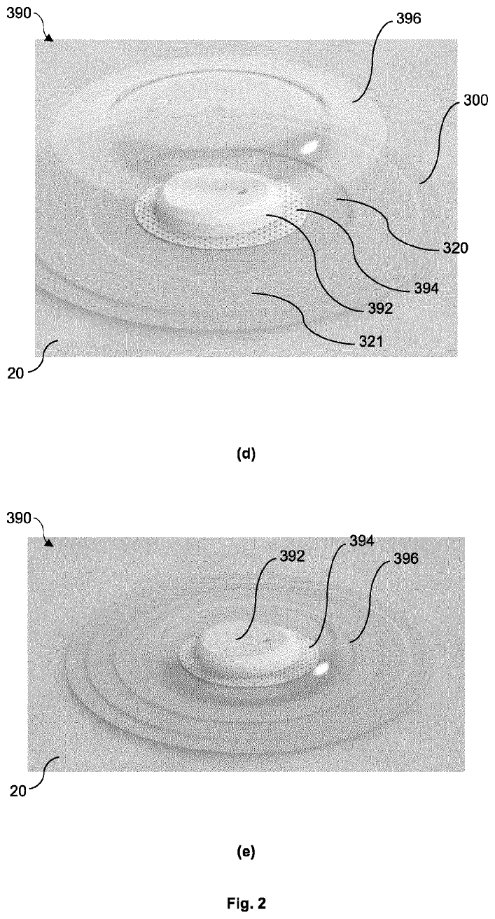

FIG. 2 shows a further example interface patch 300. The interface patch 300 has a proximal side 302 and a distal side 304 opposite the proximal side 302. The proximal side 302 includes an adhesive region. In this example, the interface patch 300 is annular in shape, having a circular outer profile and an aperture 306 in the middle.

The interface patch 300 is made from a flexible material, for example any material suitable for use as a medical patch, such as medical tapes. In examples, the interface patch 300 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

In some examples, instead of an aperture 306 the interface patch 300 may comprise a pierceable region able to be pierced by the needle of the subcutaneous medical device as it is applied to the interface patch 300. The pierceable region may be formed of thinned section of material, or may comprise a different material to the rest of the interface patch 300.

As shown in FIG. 2(b), the proximal side 302 is provided with first and second release layers 308, 310 that cover an adhesive region on the proximal side 302. The release layers 308, 310 are removable to expose the adhesive region to attach the proximal side 302 of the interface patch 300 to the patient's skin 20. As shown in FIG. 2(b), the first release layer 308 is integrally formed with a release tab 312 that protrudes outward. The second release layer 310 is integrally formed with another release tab 314 that protrudes outward and is adjacent the release tab 312. The release tabs 312, 314 may be pulled or peeled away to remove the respective release layer 308, 310 from the proximal side 302 of the interface patch 300 to expose the adhesive region for sticking the interface patch 300 onto the skin 20. In some examples, the interface patch 300 may have a single part release layer that covers the proximal side 302 of the interface patch 300.

In this example, as shown in FIGS. 2(a) to 2(c), the distal side 304 of the interface patch 300 is provided with a locating edge 322. In particular, the distal side 304 has a region with a raised surface 321 inward of the edge of the interface patch 300. The raised surface 321 provides a relative height difference across different regions of the interface patch 300. The distal side 304 has a recessed surface 320 inward of the raised surface 321 towards the aperture 306. The locating edge 322 is defined at the boundary of the raised surface 321 and the recessed surface 320. Thus, the distal side 304 of the interface patch 300 has a locating edge 322 around the aperture 306 and a circular raised portion 321 outward of the recessed surface 320 towards the edge of the interface patch 300. The locating edge 322 can be used to align the applicator (10, see FIG. 1(d)), in particular the needle (98, see FIG. 1(a)) of the subcutaneous medical device 392 with the aperture 306 when applying the subcutaneous device 392 to the interface patch 300. In particular, a circular part of the applicator can be received against the recessed surface 320, and possibly against the locating edge 322, to align the subcutaneous medical device 392 with the aperture 306. In this example the locating edge 322 is circular, but in various examples it will be appreciated that the shape and size of the locating edge 322, recessed surface 320 and raised surface 321 can be adapted to match different applicators.

As shown in FIGS. 2(d) and 2(e), a cover 396 may optionally be provided to place over the subcutaneous medical device 392 to protect the device 392 from knocking or contamination. In this example, the cover 396 is transparent to view the subcutaneous medical device 392. The cover 396 may be adhered to the interface patch 300, and in particular to the raised surface 321 as shown in FIGS. 2(d) and 2(e). Preferably, the cover 396 is rigid. The cover 396 may comprise any one or more of polyethylene, polypropylene, polyvinyl alcohol, polyurethane, or a thermoplastic elastomer, for example.

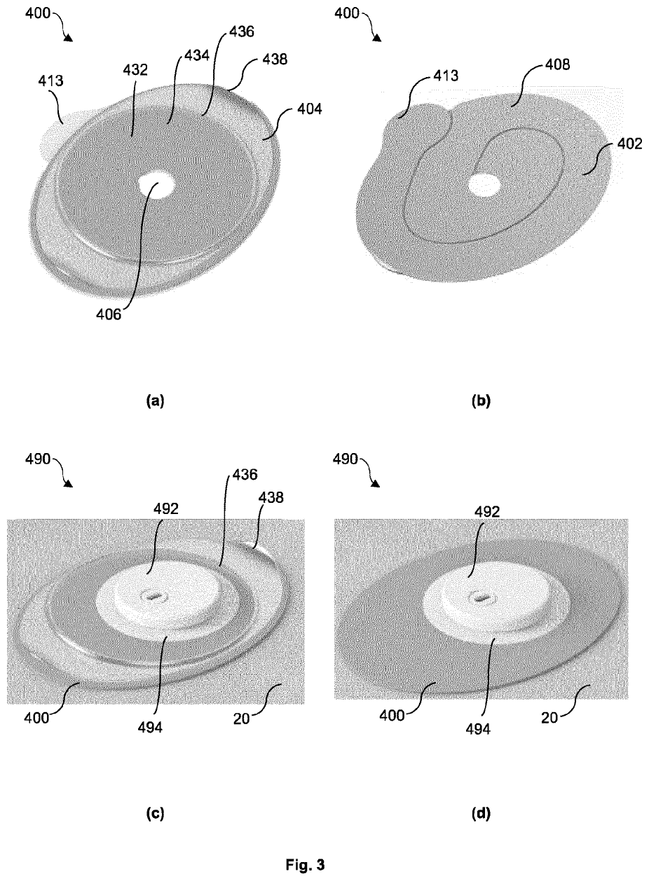

FIG. 3 shows an alternative example of an interface patch 400 that facilitates the alignment of an applicator relative to the interface patch 400. As shown in FIG. 3(b), the interface patch 400 has a proximal side 402. As shown in FIG. 3(a), the interface patch 400 has a distal side 404 opposite the proximal side 402. The proximal side 402 includes an adhesive region. The interface patch 400 is made from a flexible material, for example any material suitable for use as a medical patch, such as medical tapes. In examples, the interface patch 400 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

The proximal side 402 has a release layer 408 that is provided to cover an adhesive region on the interface patch 400. In this example, the release layer 408 has a spiral profile, but it will be appreciated that the release layer 408 may have alternative shapes and configurations, for example a single sheet or two parts as shown in FIG. 2. At the end of the release layer 408, there is provided a pull tab 413 which extends outward of the proximal side 402. The pull tab 413 can be used to remove the release layer 408 to expose the adhesive region on the proximal side 402. The interface patch 400 can then be adhered to the skin 20 by the adhesive on the proximal side 402 of the interface patch 400.

As best shown in FIG. 3(a), a support 432 is provided on the distal side 404 of the interface patch 400. The support 432 is preferably rigid. The support 432 is attached, for example adhered, to the distal side 404 of the interface patch 400. The support 432 holds the interface patch 400 in a flat position during application of the interface patch 400 to the user's skin, helping to avoid wrinkles and folds in the interface patch 400.

In addition, as illustrated in FIGS. 3(a) and 3(c), the support 432 includes an opening 434 having an edge 436. The opening 434 surrounds the aperture 406 in the interface patch 400 and allows application of a subcutaneous medical device 492 to the interface patch 400 through the opening 434 using an applicator as described with reference to FIG. 1(d). The edge 436 may serve as a locating edge for the applicator to ensure alignment of the applicator and the aperture 406 so that the needle of the subcutaneous medical device is aligned with the aperture 406. In this example the opening 434 and edge 436 are circular, but it will be appreciated that other shapes and sizes may be provided for different applicators. The edge 436 may be formed by the thickness of the support 432, or it may include a lip or protrusion.

As shown in FIG. 3(d), after application of the subcutaneous medical device 492 the support 432 can be removed, for example by pulling on the tab 438 shown in FIGS. 3(a) and 3(c). The support 432 can be adhered to the interface patch 400 using a single-use, non-tacky adhesive that offers a low adhesion force, so the support 432 can be separated from the interface patch 400 without disturbing the adhesion of the interface patch 400 to the skin 20. Alternatively, the support 432 can be adhered to the interface patch 400 using an adhesive that remains on the support 432 when the support 432 is removed from the interface patch 400.

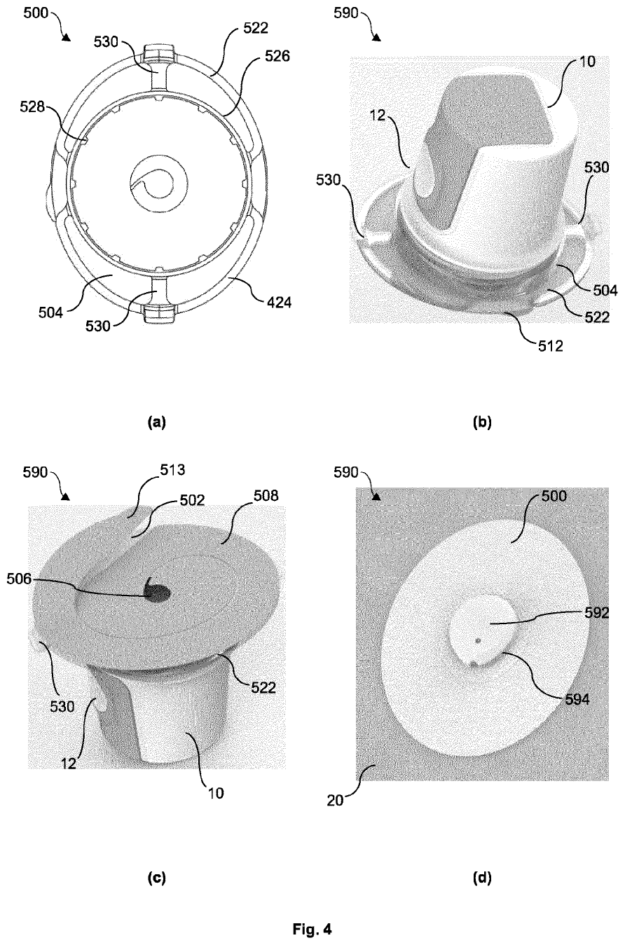

Referring now to FIG. 4, there is shown a further example interface patch 500 that can be attached to an applicator 10 before application to the patient. The interface patch 500 includes a proximal side having an adhesive region for attaching the interface patch 500 to the patient's skin. The applicator 10 is attachable to a distal side of the interface patch 500, opposite to the proximal side. The interface patch 500 is made from a flexible material, for example any material suitable for use as a medical patch, such as medical tapes. In examples, the interface patch 500 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

In this example, the interface patch 500 is provided with a support structure 522 which is attachable to the interface patch 500 and also attachable to an applicator 10 for applying the subcutaneous medical device 592.

As shown in FIGS. 4(a) and 4(b), the support structure 522 has a frame 524 sized and shaped corresponding with the interface patch 500. In this example, the interface patch 500 and the frame 524 are elliptical, but it will be appreciated that the interface patch 500 and frame 524 may be differently shaped. The interface patch 500 is attached to one side of the support structure 522, for example by an adhesive (preferably a single-use, non-tacky adhesive). In examples, the support structure 522 is adhered to the interface patch 500 using an adhesive that remains on the support structure 522 when the support structure 522 is removed from the interface patch 500. The support structure 522 is also provided with tabs 530 for peeling the support structure 522 away from the interface patch 500 after application as described below. The support structure 522 includes a circular mount 526 for engaging the applicator 10. In this example the mount 526 is provided with resilient members 528 that protrude inwardly to resiliently engage the applicator 10 and hold the support structure 522 (and interface patch 500) on the applicator 10. In other examples, the mount 526 may be a push fit onto the applicator 10, or may be attached to the applicator 10 using a fastener. When the support structure 522 is attached to the applicator 10 the subcutaneous medical device within the applicator 10 is aligned with the aperture 506 in the interface patch 500.

As shown in FIG. 4(c), the proximal side 502 of the interface patch 500 has an adhesive region that is covered by a release layer 508, which in this example is a single piece release layer having a spiral form. However, it will be appreciated that other shapes of release layer 508 may alternatively be provided.

FIGS. 4(b) to 4(d) illustrate the method of using the interface patch 500. Firstly, the interface patch 500 is attached to the support structure 522. The interface patch 500 and support structure 522 may be provided pre-assembled, or the patient may attach the interface patch 500 to the support structure 522.

Next, the support structure 522 (and interface patch 500) is attached to the applicator 10. In some examples, the patient attaches the support structure 522 to the applicator 10, but in other examples they are provided pre-assembled. To attach the applicator 10 and support structure 522 an end of the applicator 10 is pushed into the mount 526 so that the resilient members 528 engage the applicator 10.

After the applicator 10 is secured in place relative to the interface patch 500, the release layer 508 is removed from the proximal side 502 of the interface patch 500 to expose the adhesive region for securing the assembly 590 onto the patient's skin 20. The assembly 590 is then applied to the skin with the adhesive region on the interface patch 500 being pressed onto the skin.

After the assembly 590, including the interface patch 500, applicator 10 and support structure 522, is attached to the skin 20 by the adhesive region on the proximal side 502 of the interface patch 500, the actuator 12 is operated to insert the needle of the subcutaneous medical device 592 into the skin 20. The subcutaneous medical device 592 is applied in this way to the distal side 504 of the interface patch 500 such that the needle of the subcutaneous medical device 592 aligns with the aperture 506 of the interface patch 500. The adhesive patch 594 of the subcutaneous medical device 592 adheres around the aperture 506 to secure the subcutaneous medical device 592 to the interface patch 500. By attaching the applicator 10 to the interface patch 500 before attachment of the interface patch 500 to the skin 20, the need to align the applicator 10 to the interface patch 500 after attachment is removed. This is particularly advantageous if the interface patch 500 and the subcutaneous medical device 592 need to be attached to the patient's skin 20 at a location that is hard to see or hard to reach, for example the back of the arm.

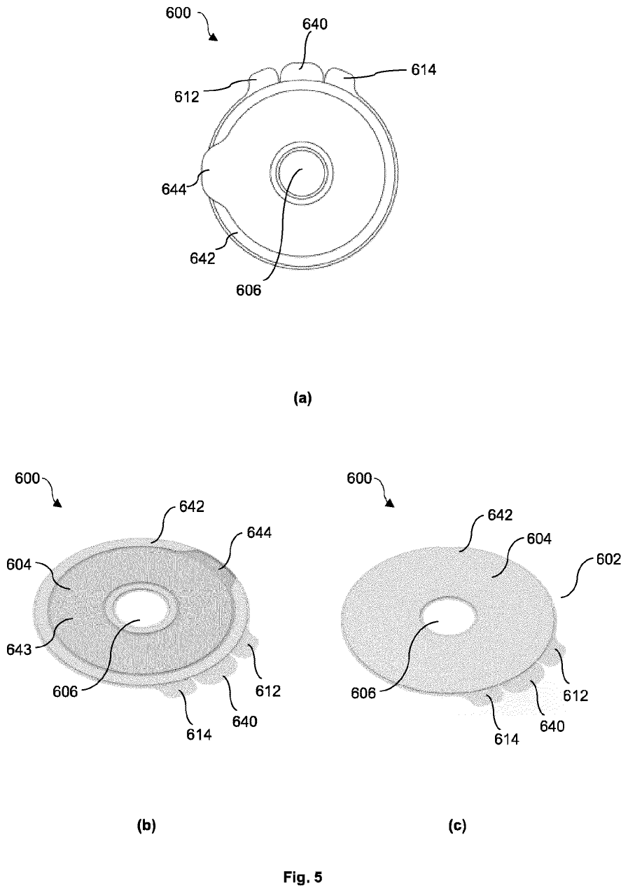

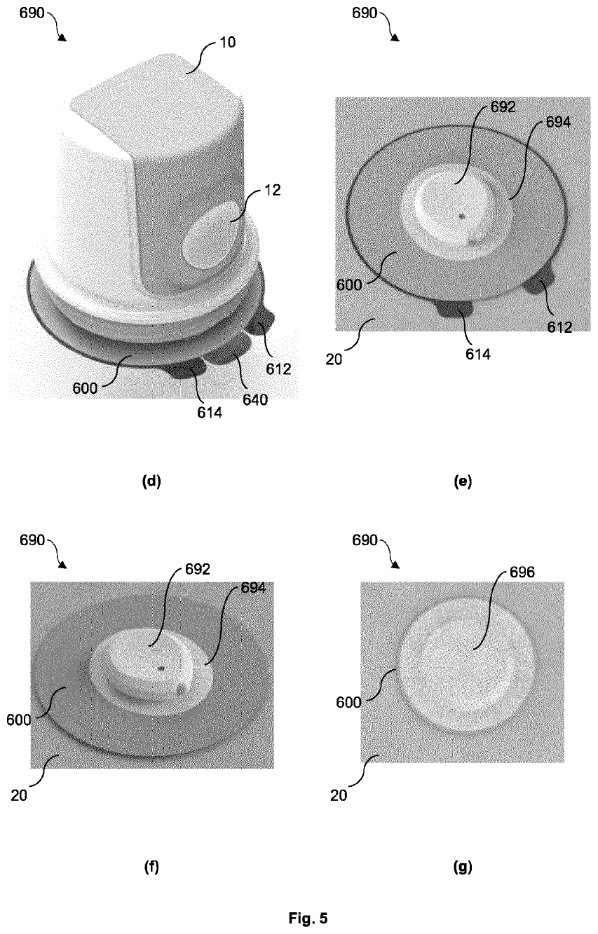

FIG. 5 shows a further example interface patch 600. In this example the interface patch 600 can be mounted to the applicator 10 before the interface patch 600 is attached to the patient's skin 20. The interface patch 600 includes a distal side 604 as shown in FIGS. 5(a), 5(b) and 5(c), and a proximal side 602 on the opposite side. In this example, the proximal side 602 is provided with an adhesive region for adhering the interface patch 600 to the patient's skin. The interface patch 600 is made from a flexible material, for example any material suitable for use as a medical patch, such as medical tapes. In examples, the interface patch 600 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

The adhesive region on the proximal side 602 is covered by a release layer (not shown) that may be the same as described with reference to FIG. 5. In particular, the release layer may have a central portion with a pull tab 640, and a peripheral portion with pull tabs 612, 614. The release layer on the proximal side 602 can be removed by pulling the pull tabs 640, 612, 614. However, it will be appreciated that various release layers may be provided, including a single sheet or two parts as shown in FIG. 4.

A central portion of the distal side 604 is provided with an adhesive region. A release layer 643 covers the central adhesive region. The adhesive may comprise a medical adhesive, for example a pressure-sensitive adhesive, such as pressure-sensitive silicone or hydrocolloid. The adhesive may comprise acrylic. The release layer 643 in this example is integrally formed with a central release layer tab 644 that can be pulled to remove the central release layer 643. A peripheral edge 642 of the distal side 604 is adhesive-free.

The method of using the interface patch 600 is best illustrated in FIGS. 5(b) to 5(f). Firstly, the release layer 643 is removed from the distal side 604 by pulling on the release tab 644 to expose an adhesive surface below the release layer 643 on the distal side 604 of the interface patch 600. With the adhesive surface exposed, an applicator 10 is attached to the distal side 604 of the interface patch 600 by the adhesive as shown in FIG. 5(d). The adhesive can adhere to an end face of the applicator 10.

The release tab 640 is then pulled to peel off the central portion of the release layer (not shown) on the proximal side 602 of the interface patch 600. By peeling off the release layer on the proximal side 602, the adhesive region is exposed for attaching the proximal side 602 to the skin 20. The assembly 690, including the applicator 10 and the interface patch 600, can now be attached to the patient's skin 20. Once the proximal side 602 is attached to the skin 20, the applicator 10 is actuated using the actuator 12 to insert the needle of the subcutaneous medical device 692 into the skin 20. The adhesive patch 694 of the subcutaneous medical device 692 adheres to the distal side 604 of the interface patch 600. After the subcutaneous medical device 692 is attached to the interface patch 600, the applicator 10 is removed and the remaining release tabs 612, 614 are pulled to peel off any remaining release layers on the proximal side 602.

As shown in FIG. 5(g), optionally a cover 696 may be provided to protect the device 692 from knocking or contamination. The cover 696 can be adhered to the interface patch 600 using the adhesive on the distal side 604 that was used to attach the applicator 10. The cover 696 may comprise any one or more of polyethylene, polypropylene, polyvinyl alcohol, polyurethane, or a thermoplastic elastomer, for example.

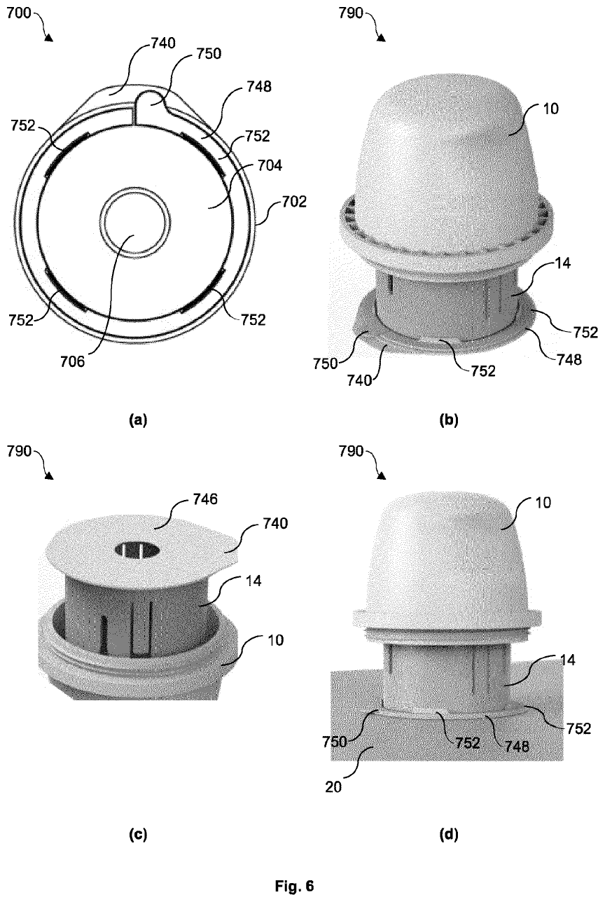

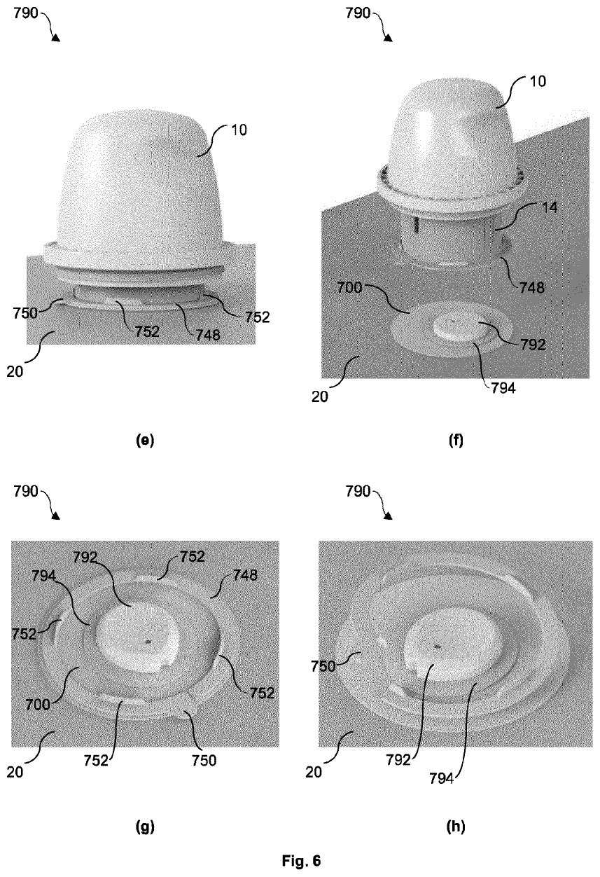

FIG. 6 shows a further example interface patch 700 that is attachable to the applicator 10 before attaching the interface patch 700 to the patient's skin 20. The distal side 704 of the interface patch 700 is shown in FIG. 6(a). The applicator 10 is attachable to the distal side 704 of the interface patch 700 as shown in FIG. 6(b). The proximal side of the interface patch 700 includes an adhesive region for adhering the interface patch 700 to the patient's skin. The interface patch 700 is made from a flexible material, for example any material suitable for use as a medical patch, such as medical tapes. In examples, the interface patch 700 may comprise polyethylene, vinyl, foam, woven or non-woven acrylic, woven or non-woven polyester, urethanes, or rayon acetate woven fabric. The adhesive of the adhesive region may comprise a medical adhesive, for example a pressure-sensitive adhesive, such as pressure-sensitive silicone or hydrocolloid. The adhesive may comprise acrylic.

In this example, the interface patch 700 includes a support structure 748 on the distal side 704 of the interface patch 700 for attaching the applicator 10 to the interface patch 700. The support structure 748 extends along the edge of the interface patch 700. In this example, the support structure 748 is annular in shape. The support structure 748 forms a tape (e.g. a semi-rigid support liner) extending about the edge of the interface patch 700 and includes a tab 750 integrally formed with the support structure 748. The support structure 748 has an outer ring edge which is sized and shape to correspond with the edge of the interface patch 700, and an inner ring edge towards the aperture 706 of the interface patch 700. The support structure 748 is adhered to the distal side 704 of the interface patch 700, preferably with a single-use, non-tacky adhesive. Alternatively, the support structure 748 is adhered to the interface patch 700 using an adhesive that remains on the support structure 748 when the support structure 748 is removed from the interface patch 700. The inner edge of the support structure 748 is provided with four support arms 752 equidistantly spaced around the inner edge that engage with the body 14 of the applicator 10 in use. The support arms 752 act to clip onto the body 14 of the applicator 10, or grip the body 14 of the applicator 10. The supports arms 752 may be resiliently deformable. When the support structure 748 is attached to the applicator 10 the subcutaneous medical device within the applicator 10 is aligned with the aperture 706 in the interface patch 700.

The proximal side 702 of the interface patch 700 is provided with a release layer 746 that covers an adhesive region on the proximal side 702, and includes a release tab 740. The adhesive region on the proximal side 702 is used to adhere the interface patch 700 to the patient's skin,

FIGS. 6(b) to 6(h) illustrate the method of using the interface patch 700. The interface patch 700 and support structure 748 may be provided pre-assembled (with the support structure 748 adhered to the distal side 704 of the interface patch 700), or the patient may adhere the support structure 748 to the interface patch in the position shown in FIG. 6(a). The device body 14 of the applicator 10 is attached to the interface patch 700 using the support structure 748. In this example, the support arms 752 are resiliently biased against the device body 14 to firmly hold the applicator 10 in place. The interface patch 700 and the applicator 10 may be pre-assembled, or the patient may attach the interface patch 700 to the applicator 10 using the support structure 748. As shown in FIG. 6(c), the release layer 746 on the proximal side 702 of the interface patch 700 is removed by pulling on the release tab 740 to peel the release layer 746 off, exposing the adhesive region. The assembly 790 including the applicator 10 and the interface patch 700 is then attached to the skin 20, as shown in FIG. 6(d). As shown in FIG. 6(e), the applicator 10 is used to apply the subcutaneous medical device 792. In this example, the applicator 10 is actuated by pushing the head of the applicator 10 downwards to collapse the device body 14 into the head, but it will be appreciated that other types of applicator 10 may be used, for example button-actuated applicators. The subcutaneous medical device 792 is applied such that the needle of the subcutaneous medical device 792 pierces into the skin 20 of the patient. At the same time, the adhesive patch 794 of the subcutaneous medical device 792 adheres around the aperture 706 to secure the subcutaneous medical device 792 to the interface patch 700.

As shown in FIG. 6(f), the applicator 10 can be removed from the interface patch 700 together with the support structure 748. Alternatively, as shown in FIGS. 6(g) and 4(h), the applicator 10 can be removed on its own from the interface patch 700, and then the support structure 748 can be subsequently removed after the removal of the applicator 10. The tab 750 can be pulled to support the removal of the support structure 748. Thus, the applicator 10 is first assembled with the interface patch 700, before the assembly 790 is attached the skin 20. This removes the need to align the applicator 10 with the interface patch 700, which is particularly beneficial if the interface patch 700 is placed in a location that is hard to reach or see, for example the back of the arm

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

Through the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible.

1. A subcutaneous medical device system, comprising an interface patch and a subcutaneous medical device having an adhesive surface and a needle for piercing a patient's skin, wherein the interface patch comprises:
a proximal side having an adhesive region for attaching the interface patch to the patient's skin; and
a distal side having a locating edge for aligning an applicator operable to apply the subcutaneous medical device to the distal side of the interface patch.

2. A subcutaneous medical device system according to claim 1, further comprising an aperture positioned to receive the needle of the subcutaneous medical device.

3. A subcutaneous medical device system according to claim 1 or claim 2, wherein the locating edge comprises a step.

4. A subcutaneous medical device system according to any one of claims 1 to 3, wherein the distal side comprises a first portion and a second portion.

5. A subcutaneous medical device system according to claim 4, wherein the locating edge is provided between the first portion and the second portion.

6. A subcutaneous medical device system according to claim 4 or claim 5, wherein the second portion is provided outward of the first portion.

7. A subcutaneous medical device system according to any one of claims 4 to 6, wherein the first portion is raised relative to the second portion.

8. A subcutaneous medical device system according to any one of claims 4 to 6, wherein the second portion is raised relative to the first portion.

9. A subcutaneous medical device system according to any one of claims 4 to 8, wherein the first portion is central on the interface patch.

10. A subcutaneous medical device system according to any one of claims 4 to 9, wherein the first portion is concentric with the second portion.

11. A subcutaneous medical device system according to any one of claims 4 to 10, further comprising a third portion outward of the second portion.

12. A subcutaneous medical device system according to claim 11, wherein an interface between the second portion and the third portion comprises an outer locating edge.

13. A subcutaneous medical device system according to claim 11 or claim 12, wherein the height of the third portion is substantially the same as the height of the first portion.

14. A subcutaneous medical device system according to any one of claims 11 to 13, wherein the third portion and the first portion are concentric.

15. A subcutaneous medical device system according to any one of claims 11 to 14, wherein the third portion and the second portion are concentric.

16. A subcutaneous medical device system according to any one of claims 11 to 15, wherein the second portion is raised relative to the third portion.

17. A subcutaneous medical device system according to any one of the preceding claims, wherein the locating edge is circular.

18. A subcutaneous medical device system according to any one of the preceding claims, comprising a support attached to the distal side of the interface patch, the support comprising the locating edge.

19. A subcutaneous medical device system according to claim 18, wherein the support is removably attached to the interface patch.

20. A subcutaneous medical device system according to claim 18 or claim 19, wherein the support is rigid.

21. A subcutaneous medical device system according to any one of the preceding claims, further comprising an applicator operable to apply the subcutaneous medical device to the interface patch.

22. A subcutaneous medical device system according to claim 21, wherein the applicator comprises an end face positionable on the interface patch, and wherein the locating edge is shaped to match the end face.

23. A subcutaneous medical device system according to any one of the preceding claims, further comprising a cover positionable over the subcutaneous medical device and comprising a concave portion for accommodating the subcutaneous medical device.

24. A subcutaneous medical device system, comprising:
a subcutaneous medical device comprising an adhesive surface and a needle for piercing a patient's skin;
an applicator operable to apply the subcutaneous medical device; and
an interface patch comprising a proximal side having an adhesive for attaching the interface patch to the patient's skin, and a distal side to which the adhesive surface of the subcutaneous medical device is attachable,
wherein the interface patch is attachable to the applicator before operation of the applicator.

25. A subcutaneous medical device system according to claim 24, wherein the interface patch is adherable to the applicator.

26. A subcutaneous medical device system according to claim 25, wherein the distal side of the interface patch comprises an adhesive portion for adhering the interface patch to the applicator.

27. A subcutaneous medical device system according to claim 26, wherein the adhesive portion covers substantially the entire distal side of the interface patch.

28. A subcutaneous medical device system according to claim 26, wherein the adhesive portion comprises a pattern-applied adhesive.

29. A subcutaneous medical device system according to any one of claims 24 to 28, wherein the applicator comprises an end face arranged to contact the distal side of the interface patch.

30. A subcutaneous medical device system according to claim 24, wherein the interface patch comprises a support structure for attaching the interface patch to the applicator.

31. A subcutaneous medical device system according to claim 30, wherein the support structure comprises an outer portion.

32. A subcutaneous medical device system according to claim 31, wherein the outer portion of the support structure is coincident with the edge of the interface patch.

33. A subcutaneous medical device system to any one of claims 30 to 32, wherein the support structure comprises an inner portion.

34. A subcutaneous medical device system according to claim 33, wherein the inner portion is attachable to the applicator.

35. A subcutaneous medical device system according to claim 33 or claim 34, wherein the inner portion of the support structure has a circular shape.

36. A subcutaneous medical device system according to any one of claims 33 to 35, wherein the inner portion of the support structure comprises at least one support member configured for engaging the applicator.

37. A subcutaneous medical device system according to claim 36, wherein the support member resiliently biases against the applicator during use.

38. A subcutaneous medical device system according to any one of claims 24 to 37, wherein the interface patch comprises an aperture aligned with the needle of the subcutaneous medical device when the interface patch is attached to the applicator, and optionally wherein the aperture is arranged substantially in the center of the interface patch.

39. A subcutaneous medical device system according to any one of claims 24 to 37, wherein the interface patch comprises a pierceable region aligned with the needle of the subcutaneous medical device when the interface patch is attached to the applicator.

40. A subcutaneous medical device system according to claim 39, wherein the pierceable region is arranged substantially in the center of the interface patch.

41. A subcutaneous medical device system according to claim 39 or claim 40, wherein the pierceable region comprises thinned section of the interface patch.

42. A subcutaneous medical device system according to any one of claims 24 to 41, wherein the interface patch has a generally circular shape.

43. A subcutaneous medical device system according to any one of claims 24 to 42, wherein the subcutaneous medical device is a glucose sensor, an intermittent glucose sensor, a continuous glucose sensor, or an infusion set.

44. A support structure for attaching an interface patch for a subcutaneous medical device to an applicator operable to apply the subcutaneous medical device to a patient, the support structure comprising a first surface for attaching to a distal side of the interface patch, and a mount for attachment to the applicator.
